SEQUENCE LISTING (62 sequences)

SEQ ID NO: 1 - DNA, 18 bp, Artificial Sequence
  B11/F3
ttttcctgctggtggctc

SEQ ID NO: 2 - DNA, 20 bp, Artificial Sequence
  B11/B3
tgagagaagtccaccacgag

SEQ ID NO: 3 - DNA, 40 bp, Artificial Sequence
  B11/FIP
cggtcctcgcggagattgacggaacagtaaaccctgctcc

SEQ ID NO: 4 - DNA, 40 bp, Artificial Sequence
  B11/BIP
taggacccctgcccgtgttagactctgcggtattgtgagg

SEQ ID NO: 5 - DNA, 21 bp, Artificial Sequence
  B11/LF
ggcggggtttttcttgttgac

SEQ ID NO: 6 - DNA, 20 bp, Artificial Sequence
  B11/LB
tcctcacaataccgcagagt

SEQ ID NO: 7 - DNA, 20 bp, Artificial Sequence
  B12/F3
tcctcacaataccgcagagt

SEQ ID NO: 8 - DNA, 20 bp, Artificial Sequence
  B12/B3
gcagcaggatgaagaggaat

SEQ ID NO: 9 - DNA, 42 bp, Artificial Sequence
  B12/FIP
cgcgaattttggccaagacacatagactcgtggtggacttct

SEQ ID NO: 10 - DNA, 40 bp, Artificial Sequence
  B12/BIP
tcactcaccaacctcctgtcctaaaacgccgcagacacat

SEQ ID NO: 11 - DNA, 23 bp, Artificial Sequence
  B12/LF
ggtgatccccctagaaaattgag

SEQ ID NO: 12 - DNA, 23 bp, Artificial Sequence
  B12/LB
caatttgtcctggttatcgctgg

SEQ ID NO: 13 - DNA, 19 bp, Artificial Sequence
  B13/F3
gtggctccagttcaggaac

SEQ ID NO: 14 - DNA, 19 bp, Artificial Sequence
  B13/B3
agaagtccaccacgagtct

SEQ ID NO: 15 - DNA, 40 bp, Artificial Sequence
  B13/FIP
ccatgttcgtcacagggtcccaccctgctccgaatattgc

SEQ ID NO: 16 - DNA, 40 bp, Artificial Sequence
  B13/BIP
taggacccctgcccgtgttaactctgcggtattgtgagga

SEQ ID NO: 17 - DNA, 23 bp, Artificial Sequence
  B13/LF
gcggagattgacgagatgtgaga

SEQ ID NO: 18 - DNA, 21 bp, Artificial Sequence
  B13/LB
ggcggggtttttcttgttgac

SEQ ID NO: 19 - DNA, 18 bp, Artificial Sequence
  B14/F3
cgtcttgggctttcgcaa

SEQ ID NO: 20 - DNA, 18 bp, Artificial Sequence
  B14/B3
gcagagcttggtggaagg

SEQ ID NO: 21 - DNA, 40 bp, Artificial Sequence
  B14/FIP
ggaaagccctacgaaccactgatgggcctcagtccgtttc

SEQ ID NO: 22 - DNA, 41 bp, Artificial Sequence
  B14/BIP
gatgatgtggtattgggggccatggaaggggtttacctcgg

SEQ ID NO: 23 - DNA, 21 bp, Artificial Sequence
  B14/LF
tggcactagtaaactgagcca

SEQ ID NO: 24 - DNA, 23 bp, Artificial Sequence
  B14/LB
gtctgtacagcatcgtcatgaca

SEQ ID NO: 25 - DNA, 19 bp, Artificial Sequence
  B15/F3
catctcgtcaatctccgcg

SEQ ID NO: 26 - DNA, 19 bp, Artificial Sequence
  B15/B3
tggggatcgcgaattttgg

SEQ ID NO: 27 - DNA, 38 bp, Artificial Sequence
  B15/FIP
aacacgggcaggggtcctaggggaccctgtgacgaaca

SEQ ID NO: 28 - DNA, 41 bp, Artificial Sequence
  B15/BIP
accgcagagtctagactcgtggccaagacacacgggtgatc

SEQ ID NO: 29 - DNA, 21 bp, Artificial Sequence
  BA/F3
tcctcacaataccacagagtc

SEQ ID NO: 30 - DNA, 23 bp, Artificial Sequence
  BA/B3
ccagaagaaccaacaagaagatg

SEQ ID NO: 31 - DNA, 43 bp, Artificial Sequence
  BA/FIP
ggaggttggggactgcgaatttttagcacccacgtgtcctggc

SEQ ID NO: 32 - DNA, 38 bp, Artificial Sequence
  BA/BIP
ccagggttcacccctccacaacactggggtcaacatgc

SEQ ID NO: 33 - DNA, 20 bp, Artificial Sequence
  B41/F3
gcgggtcaccatattcttgg

SEQ ID NO: 34 - DNA, 18 bp, Artificial Sequence
  B41/B3
tgctcccactcctacctg

SEQ ID NO: 35 - DNA, 41 bp, Artificial Sequence
  B41/FIP
tcccagagggttgggaacagaagagctacagcatgggaggt

SEQ ID NO: 36 - DNA, 42 bp, Artificial Sequence
  B41/BIP
gttggaccctgtattcggagccggtccttgatggggttgaag

SEQ ID NO: 37 - DNA, 20 bp, Artificial Sequence
  B41/LF
gtccccatgcctttgcgagg

SEQ ID NO: 38 - DNA, 20 bp, Artificial Sequence
  B42/F3
attcggagccaactcaaaca

SEQ ID NO: 39 - DNA, 19 bp, Artificial Sequence
  B42/B3
ggaggcaggaggaggaatt

SEQ ID NO: 40 - DNA, 41 bp, Artificial Sequence
  B42/FIP
tgctcccactcctacctggttttgggacttcaaccccatca

SEQ ID NO: 41 - DNA, 38 bp, Artificial Sequence; identical to SEQ ID NO: 32
  B42/BIP

SEQ ID NO: 42 - DNA, 20 bp, Artificial Sequence
  B42/LB
tgttttggggtggagccctc

SEQ ID NO: 43 - DNA, 19 bp, Artificial Sequence
  B43/F3
tcaaccccatcaaggacca

SEQ ID NO: 44 - DNA, 20 bp, Artificial Sequence
  B43/B3
gcctgaggatgactgtctct

SEQ ID NO: 45 - DNA, 39 bp, Artificial Sequence
  B43/FIP
ccaaaacaccgccgtgtggaagccaaccaggtaggagtg

SEQ ID NO: 46 - DNA, 39 bp, Artificial Sequence
  B43/BIP
caggctcagggcatgttgacctaggctgccttcctgact

SEQ ID NO: 47 - DNA, 19 bp, Artificial Sequence
  B43/LF
aaccctggcccgaatgctc

SEQ ID NO: 48 - DNA, 22 bp, Artificial Sequence
  B43/LB
gtcaacaattcctcctcctgcc

SEQ ID NO: 49 - DNA, 20 bp, Artificial Sequence
  B44/F3
actctttggaaggcgggtat

SEQ ID NO: 50 - DNA, 20 bp, Artificial Sequence
  B44/B3
ttgtttgagttggctccgaa

SEQ ID NO: 51 - DNA, 42 bp, Artificial Sequence
  B44/FIP
accaacctcccatgctgtagctgagagaaaccacacgtagcg

SEQ ID NO: 52 - DNA, 39 bp, Artificial Sequence
  B44/BIP
cctcgcaaaggcatggggacgggtccaactgatgatcgg

SEQ ID NO: 53 - DNA, 24 bp, Artificial Sequence
  B44/LF
cccaagaatatggtgacccgcaaa

SEQ ID NO: 54 - DNA, 21 bp, Artificial Sequence
  B44/LB
ccaaccctctgggattctttc

SEQ ID NO: 55 - DNA, 19 bp, Artificial Sequence
  B45/F3
tcccaaccctctgggattc

SEQ ID NO: 56 - DNA, 18 bp, Artificial Sequence
  B45/B3
cactggggtcaacatgcc

SEQ ID NO: 57 - DNA, 42 bp, Artificial Sequence
  B45/FIP
ccttgatggggttgaagtcccaagttggaccctgtattcgga

SEQ ID NO: 58 - DNA, 38 bp, Artificial Sequence
  B45/BIP
gccagcagccaaccaggtagctccaccccaaaacaccg

SEQ ID NO: 59 - DNA, 22 bp, Artificial Sequence
  BB/F3
ggagccaactcaaacaatccag

SEQ ID NO: 60 - DNA, 21 bp, Artificial Sequence
  BB/B3
gagagatgggagtaggctgtc

SEQ ID NO: 61 - DNA, 44 bp, Artificial Sequence
  BB/FIP
gaaccctggcccgaatgctcttttgccagaggcaaatcaggtag

SEQ ID NO: 62 - DNA, 47 bp, Artificial Sequence
  BB/BIP
tggagccctcaggctcagggttttttattggtggaggcaggaggagg

(19) United States
(12) Patent Application Publication
(10) Pub. No.: US 2012/0202190 A1
(43) Pub. Date: Aug. 9, 2012

(54) DETECTION OF HEPATITIS B VIRUS IN BLOOD USING LAMP METHOD

(76) Inventors: Wei-Mei Ching, Bethesda, MD (US); Chien-Chung Chao, N. Potomac, MD (US); Hua-Wei Chen, Germantown, MD (US)

(21) Appl. No.: 13/370,283

(22) Filed: Feb. 9, 2012

Related U.S. Application Data
(60) Provisional application No. 61/441,099, filed on Feb. 9, 2011.

(51) Int. Cl. C12Q 1/70

(52) U.S. Cl. 435/5

(57) ABSTRACT

A method for detection blood borne pathogen in whole blood, comprising collecting a blood sample from a subject, heating said sample for a period of time, adding said heated sample to a pre-mixed LAMP solution comprising one or more LAMP primer set, and Bst DNA polymerase to create a reaction mixture, incubating said reaction mixture for a period of time and determining the presence of blood born pathogen.

### CROSS-REFERENCE TO RELATED APPLICATIONS

This application claims priority to U. S. Provisional Application No. 61/441,099 filed on 9 Feb. 2011.

### BACKGROUND

The present invention relates to the detection of pathogens in blood. More specifically, it relates to a new method for rapid detection of hepatitis B virus in blood. Fresh whole blood transfusion has been used to treat injured U.S soldiers since World War I. Blood supply in the battlefield is critical in reducing casualties, not only for military personnel but also for other traumatic victims. For example, 3,287 patients were treated at the 31st Combat Support Hospital in Baghdad for life threatening injuries in the year of 2004 (1). Between March 2003 and July 2007, over 6000 units of warm fresh whole blood have been transfused in Afghanistan and Iraq without the screening of blood pathogens (2). Transfusion of fresh whole blood has been shown to provide better outcome as compared to stored blood products collected in the US, and transported to Combat Support Hospital for life-threatening traumatic injuries.

Due to the short shelf life of platelets and the time required for transportation, freshly collected whole blood is preferred compare to platelet supply or other blood components that also have short shelf life. However, without a blood pathogen screening process, the risks associated with blood locally collected are unknown. It has been recognized that current screening systems are either not field deployable or not sensitive enough in detecting blood borne pathogens, such as hepatitis B virus (HBV), in the field. Improved detection of this and other blood borne pathogens will increase blood safety of blood collected in the field and used for transfusion.

Hepatitis B is caused by infection with the hepatitis B virus. The incubation period from the time of exposure to the onset of symptoms is 6 weeks to 6 months. This asymptomatic period is of particular important concern when fresh whole blood is used for transfusion (6, 7). HBV is found in the highest concentration in blood and in lower concentrations in other body fluids such as semen, vaginal secretions, and wound exudates. HBV infection can be self-limited or chronic. In adults, only approximately half of newly acquired HBV infections are symptomatic, and half are asymptomatic. This further emphasizes the importance of blood screening before transfusion use. Approximately 1% of reported HBV infection cases result in acute liver failure and death. Risk for chronic infection is inversely related to age at infection. HBV may be contracted through sexual intercourse with an infected person, and once a person becomes hepatitis B surface antigen positive, they become potentially infectious to others.

Hepatitis B is a major global public health problem. According to World Health Organization (WHO) current statistics, more than 2 billion people alive today have been infected with HBV worldwide at some time. Roughly 350 million remain chronically infected and become carriers of the virus, and 4 million new acute clinical cases occur annually (8).

As with most infectious diseases, there are geographic regions of varying prevalence. Regions that are more endemic for HBV include Southeast Asia, sub-Saharan Africa, the Amazon Basin, parts of the Middle East and some Eastern European countries. These are all regions where the US deploys military personnel both in time of war and for peacekeeping and disease surveillance purposes. Patients with life-threatening injuries that require any blood component that is not immediately available will be transfused with locally collected fresh whole blood. U.S. Army military clinical practice guidelines also suggest fresh whole blood transfusions for patients who continue to have significant bleeding with life-threatening injuries after receiving stored RBCs, plasma, and platelets in a 1:1:1 ratio (2). In the past, these blood supplies were not tested for blood pathogens at the time of transfusion because there is no rapid, simple bedside test available.

Current methods testing blood for contamination by the infectious pathogens are cumbersome in that they require specialized equipment, highly trained personnel and substantial time (days to weeks) to obtain a result. For example, current methods for detecting the presence of blood transmissible hepatitis B virus are immunoassays and nucleic acid amplification (7). The immunoassay for Hepatitis B lacks sensitivity and requires much instrumentation, such as ELISA detection of hepatitis B surface antigen (HBsAg). Furthermore, seroconversion, which is the development of detectable specific antibodies to microorganisms in blood as results of an infection, often takes few weeks to several months. The most sensitive way to detect pathogens circulating in blood is amplification of nucleic acid. The presence of viral nucleic acid is the earliest biomarker after infection. A sensitive nucleic acid based assay will reduce the window period, which is defined as the time from an infection to the time of detection of pathogenic antigens or antibodies to that pathogen. However, currently this method requires sophisticated instruments and substantial training of the end users. A rapid, robust, and easy-to-perform assay is in urgent need to improve the safety of blood supply collected locally.

Loop-mediated isothermal amplification, or LAMP, is a DNA amplification technique developed and pioneered by Notomi et al. (13). It is an auto-cycling strand displacement DNA synthesis method that can be performed at a single temperature around 60°-65° C. The Bst DNA polymerase used in LAMP provides an additional advantage for the detection in blood samples due to its insensitivity to blood components, such as myoglobin, heme-blood protein complexes and immunoglobin. These components inhibit the Taq polymerase, which is used in PCR (14). Thus much simpler sample preparation and reaction conditions are required for LAMP compared to conventional PCR or real-time, quantitative PCR.

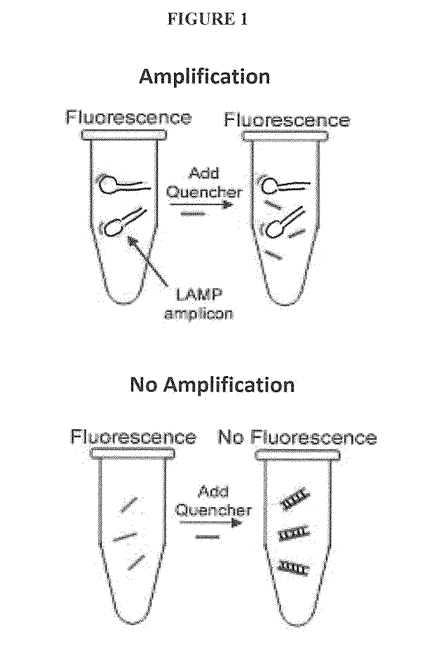

Additionally, the sensitivity of LAMP is comparable or higher than PCR. LAMP reaction can be carried out in a single tube at a fixed temperature, and takes about 30-60 minutes to generate products that can be read by the naked eye. LAMP amplification is based on the recognition of six independent sequences in the initial production phase of starting material, and four independent sequences in the later amplification phases. This specificity allows the detection of as low as six copies of a DNA target from 100 ng of genomic DNA with no significant change in background or in amplification efficiency (13). The LAMP reaction occurs in three general phases (FIG. 1 of Notomi (13)) including: an initial Starting Material Production phase, the Cyclic Amplification phase and the Elongation phase. The reaction products of LAMP are a combination of stem-loop double-stranded DNA, with variable length stems, and multiple-looped structures composed of alternating inverted repeats of the target sequence.

The sequence-specific amplification using LAMP is self-sustainable. A large amount of DNA is synthesized, yielding a significant amount of pyrophosphate ion by-product. Pyrophosphate ion combines with divalent metallic ion forms an insoluble salt. Adding manganese ion and calcein, a fluorescent metal indicator, to the reaction solution allows a visualization of substantial alteration of the fluorescence. As the fluorescence change is highly sensitive, this system enables visual discrimination of results without costly specialized equipment. Visual detection of amplification product can also be achieved with the addition of SYBR green or other intercalating fluorescent dyes.

Recent research has attempted to develop sensitive molecular test for the detection of pathogens in heated blood samples using LAMP assay (29, 30, and 31). In particular. Nagamine et al. (29) developed a method, which accelerates LAMP reaction by using additional primers. This accelerated LAMP method uses additional loop primers to achieve reaction times of less than half of the original LAMP method, allowing quick diagnosis in the clinical laboratory. Amplification of HBV DNA extracted from HBV-positive serum was used to demonstrate the sensitivity of this new method.

### DETAILED DESCRIPTION OF DRAWINGS

FIG. 1: An illustration of LAMP product detection by fluorescence.

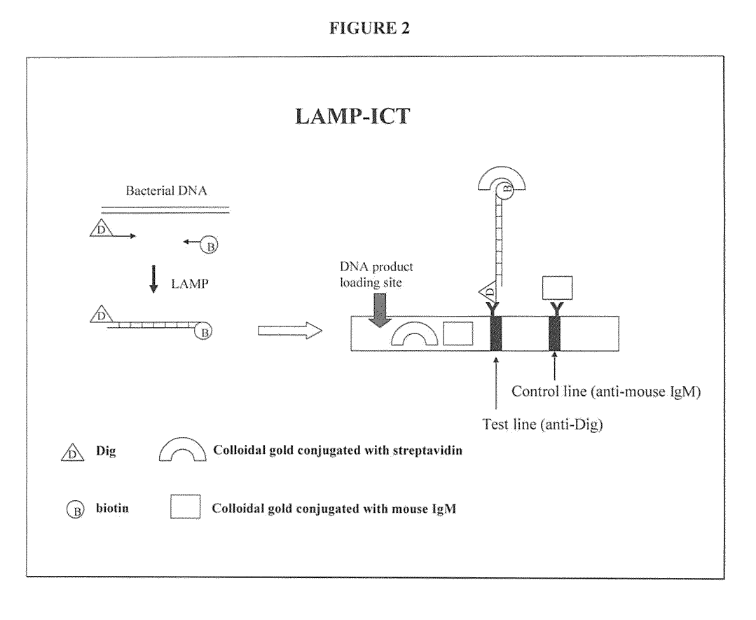

FIG. 2: An illustration of LAMP product detection by ICT.

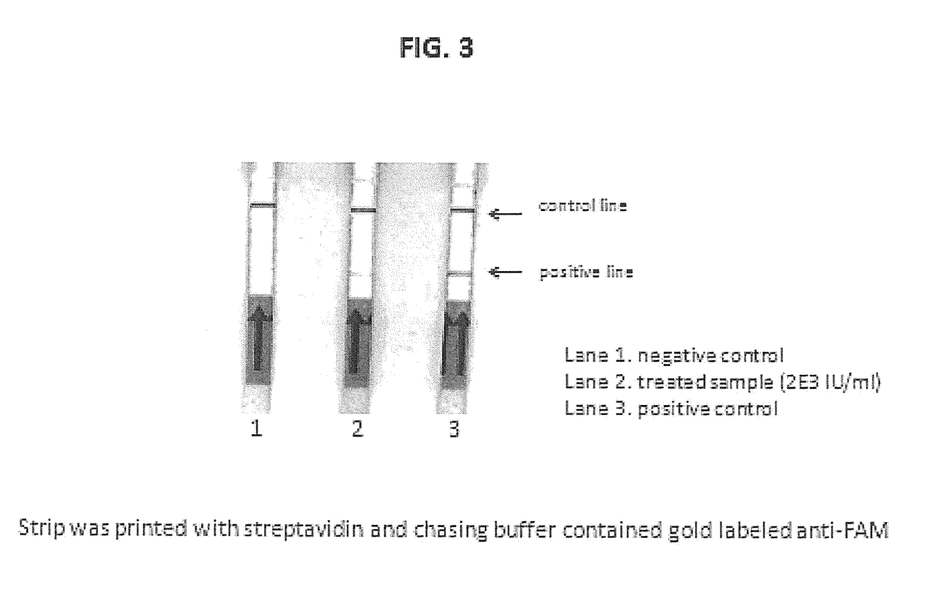

FIG. 3: Detection of HBV in heated plasma mock samples using double labeled primer set B43.

### DETAILED DESCRIPTION OF THE INVENTION

An objective of this invention is a highly sensitive and specific nucleic acid based assay that is capable of detecting HBV in blood sample. A further objective of this invention is a sensitive nucleic acid based assay for the detection of HBV from a small sample of whole blood using Loop-Mediated Isothermal Amplification (LAMP). A further objective of this invention is a fully deployable, easy to use, thermally stable and cost effective assay for blood screening in austere field condition.

A method for detecting a blood-borne pathogen in blood comprising:
(a) collecting a blood sample from a subject;
(b) heating said sample for a period of time;
(c) adding said heated sample to a pre-mixed LAMP solution creating a reaction mixture, wherein said pre-mixed LAMP solution comprising: a set of LAMP primers and Bst DNA polymerase;
(d) incubating said reaction mixture for a period of time; and
(e) detecting the presence of said blood-borne pathogen.

The blood sample may be used for the instant detection method include whole blood, plasma and serum. Once collected, the blood sample is heated at approximately 90-135° C. for approximately 5-20 minutes. In an embodiment, the collected blood sample is heated at approximately 115-125° C. for approximately 6-15 minutes. After heat treatment, a small amount of the heated blood sample is added to a pre-mixed LAMP solution to form a reaction mixture. The pre-mixed LAMP solution contains LAMP primers selected from primer sets B11, B12, B13, B14, B15, BA, B41, B42. B43, B44, B45, BB or primer set 2. The LAMP primers of these primer sets are listed in table 1. The LAMP solution may further include a buffer and water to maintain the reaction mixture at a pH of 6.0-9.5. The reaction mixture is incubated at approximately 50-80° C. for at least 15 minutes. In one embodiment, the reaction mixture is incubated at 60° C. for approximately 60 minutes. In order to determine the presence of the HBV, a fluorescent metal indicator, such as manganese ion plus calcein or SYBR Green may be added to the reaction mixture. Other intercalating fluorescent dyes may also be used. The presence of the blood borne pathogen may then be detected using a fluorescent reader.

Alternatively, as shown in FIG. 1, a fluorescent molecule, such as FAM, may be linked to the 5′ end of any primer in a LAMP primer set. After incubation, a quencher probe is added to the reaction mixture. The quencher probe consists of the complementary sequence of the selected primer tagged a quencher, such as BHQ, on its 3′ end. If target DNA is present in the blood sample, the labeled-primers are incorporated into the amplicon. The fluorescence of the incorporated labeled primers can't be quenched by the free quencher in reaction solution. The reaction mixture will remains fluorescent upon addition of the quencher probe, as the labeled-primer is no longer accessible. In the absence of a target DNA, the labeled-primer is free to bind the quencher probe, quenching the fluorescence of the reaction mixture.

In another embodiment, as illustrated in FIG. 2, the detection of the presence of said blood-borne pathogen may also be accomplished by immunochromatography (ICT). In an immunochromatography method using the sandwich method, a labeled second antibody capable of specifically binding to an analyte (for example, an antigen), and a sample solution, which may possibly contain the analyte are developed on an insoluble thin film-shaped support (for example, a glass fiber membrane, a nylon membrane, or a cellulose membrane) on which a first antibody capable of specifically binding to the analyte has been immobilized in a specific region. As a result, an immune complex with the analyte is formed at the region of the insoluble thin film-shaped carrier, on which region the first antibody has been immobilized. The analyte can be measured by detecting a signal such as color development or coloring of a label. The label to be used herein may be, for example, a protein such as an enzyme, colored latex particles, metal colloids, or carbon particles. In one embodiment, a primer from the primer set may be labeled with a tag, such as digoxigenin. After the LAMP reaction is complete, the reaction mixture is loaded onto a LAMP-ICT strip. As reaction mixture pass through the strip, amplicons that incorporated the tag-labeled primers are captured by anti-tag coated on one region of the strip, such as anti-Dig. A different primer of the primer set may be labeled with a second tag capable of capturing a label, which allows for the detecting of a signal. As shown in FIG. 2, two primers from the primer set are labeled with digoxigenin and biotin, respectively. After reaction, the reaction mixture was loaded onto an ICT, and flowing buffer is added to the sample well. As the reaction mixture pass through the strip, the presence of amplified product (HBV DNA) will be captured by anti-Dig antibody and visualized by streptavidin-colloidal gold, which is conjugated to anti-biotin. The presence of HBV in the sample can be detected by the presence or absence of a test line on the testing strip at room temperature. The ICT strip may also contain a region coated with a labeled antibody, such as a gold conjugated-Ig M or gold conjugated-IgG. The presence of a control line in a control region, which downstream from testing line and is capable of capturing said labeled antibody, indicates normal flow of the liquid through the strip. The absence of the control line due to the capture of labeled antibody indicates invalidation of the assay.

### Example 1

### Design LAMP Primers for HBV Detection

The genome of HBV is made of circular DNA. However, the DNA is not fully double-stranded. One end of the full length strand is linked to the viral DNA polymerase. The full-length strand of the HBV genome is 3020-3320 nucleotides long. The short-length strand is 1700-2800 nucleotides long (32). There are four known genes encoded by the HBV genome, called C, X, P, and S. The core protein is coded by gene C (HBcAg), and its start codon is preceded by an upstream in-frame AUG start codon from which the pre-core protein is produced. Hepatitis B envelope antigen (HBeAg) is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes are produced and named large, middle, and small (pre-S1+, pre-S2+S, pre-S2+S, or S).

The primers of table 1 were designed from the highly conserved region of pre-Surface/Surface region (2848 to 833 nt) of HBV. Two regions (˜800 nt) were chosen (1 to 800 nt and 2400 to 3220 nt) and submitted to LAMP primer designing software “PrimerExplorer” (http://primerexplorer.jp/e/). The program generates 6 primers (F3/B3, FIP/BIP, and LF/LB) for each of the LAMP primer set, targeting six different regions within a sequence of 200 to 400 nucleotides. Five sets (B11, B12, B13, B14, B15) of primers were generated in region 1 (1 to 800 nt) and five sets of primers (B41, B42, B43, B44, B45) were generated in region 2 (2400 to 3220 nt). One set of primers are manually designed from each of two conserved region without using primer explorer program (BA in region 1 and BB in region 2). These newly designed primer sets are listed in Table 1.

TABLE 1
Sequence of the newly designed primer sets.

| | | | SEQ |
|---|---|---|---|
| Set | Primer | Sequence 5′ to 3′ | ID |
| B11 | F3 | TTTCCTGCTGGTGGCTC | 1 |
| B11 | B3 | TGAGAGAAGTCCACCACGAG | 2 |
| B11 | F1P | CGGTCCTCGCGGAGATTGAC- | 3 |
| | | GGAACAGTAAACCCTGCTCC | |
| B11 | B1P | TAGGACCCCTGCCCGTGTTA- | 4 |
| | | GACTCTGCGGTATTGTGAGG | |
| B11 | LF | GGCGGGGTTTTTCTTGTTGAC | 5 |
| B11 | LB | TCCTCACAATACCGCAGAGT | 6 |
| B12 | F3 | TCCTCACAATACCGCAGAGT | 7 |
| B12 | B3 | GCAGCAGGATGAAGAGGAAT | 8 |
| B12 | F1P | CGCGAATTTTGGCCAAGACACA- | 9 |
| | | TAGACTCGTGGTGGACTTCT | |
| B12 | B1P | TCACTCACCAACCTCCTGTCCT- | 10 |
| | | AAAACGCCGCAGACACAT | |
| B12 | LF | GGTGATCCCCCTAGAAAATTGAG | 11 |
| B12 | LB | CAATTTGTCCTGGTTATCGCTGG | 12 |
| B13 | F3 | GTGGCTCCAGTTCAGGAAC | 13 |
| B13 | B3 | AGAAGTCCACCACGAGTCT | 14 |
| B13 | F1P | CCATGTTCGTCACAGGGTCCC- | 15 |
| | | ACCCTGCTCCGAATATTGC | |
| B13 | B1P | TAGGACCCCTGCCCGTGTTA- | 16 |
| | | ACTCTGCGGTATTGTGAGGA | |
| B13 | LF | GCGGAGATTGACGAGATGTGAGA | 17 |
| B13 | LB | GGCGGGGTTTTTCTTGTTGAC | 18 |
| B14 | F3 | CGTCTTGGGCTTTCGCAA | 19 |
| B14 | B3 | GCAGAGCTTGGTGGAAGG | 20 |
| B14 | F1P | GGAAAGCCCTACGAACCACTGA- | 21 |
| | | TGGGCCTCAGTCCGTTTC | |
| B14 | B1P | GATGATGTGGTATTGGGGGCCA- | 22 |
| | | TGGAAGGGGTTTACCTCGG | |
| B14 | LF | TGGCACTAGTAAACTGAGCCA | 23 |
| B14 | LB | GTCTGTACAGCATCGTCATGACA | 24 |
| B15 | F3 | CATCTCGTCAATCTCCGCG | 25 |
| B15 | B3 | TGGGGATCGCGAATTTTGG | 26 |
| B15 | F1P | AACACGGGCAGGGGTCCTAG- | 27 |
| | | GGGACCCTGTGACGAACA | |
| B15 | B1P | ACCGCAGAGTCTAGACTCGTGG- | 28 |
| | | CCAAGACACACGGGTGATC | |
| BA | F3 | TCCTCACAATACCACAGAGTC | 29 |
| BA | B3 | CCAGAAGAACCAACAAGAAGATG | 30 |
| BA | F1P | GGAGGTTGGGGACTGCGAAT TTTT | 31 |
| | | AGCACCCACGTGTCCTGGC | |
| BA | B1P | CTTGTCCTCCAATTTGTCCTG TTTTTT | 32 |
| | | GGAATATGATAAAACGCCGCAG | |
| B41 | F3 | GCGGGTCACCATATTCTTGG | 33 |
| B41 | B3 | TGCTCCCACTCCTACCTG | 34 |
| B41 | F1P | TCCCAGAGGGTTGGGAACAGAA- | 35 |
| | | GAGCTACAGCATGGGAGGT | |
| B41 | B1P | GTTGGACCCTGTATTCGGAGCC- | 36 |
| | | GGTCCTTGATGGGGTTGAAG | |
| B41 | LF | GTCCCCATGCCTTTGCGAGG | 37 |
| B42 | F3 | ATTCGGAGCCAACTCAAACA | 38 |
| B42 | B3 | GGAGGCAGGAGGAGGAATT | 39 |
| B42 | F1P | TGCTCCCACTCCTACCTGGTT- | 40 |
| | | TTGGGACTTCAACCCCATCA | |
| B42 | B1P | CCAGGGTTCACCCCTCCACA- | 41 |
| | | ACACTGGGGTCAACATGC | |
| B42 | LB | TGTTTTGGGGTGGAGCCCTC | 42 |
| B43 | F3 | TCAACCCCATCAAGGACCA | 43 |
| B43 | B3 | GCCTGAGGATGACTGTCTCT | 44 |
| B43 | F1P | CCAAAACACCGCCGTGTGGA- | 45 |
| | | AGCCAACCAGGTAGGAGTG | |
| B43 | B1P | CAGGCTCAGGGCATGTTGACC- | 46 |
| | | TAGGCTGCCTTCCTGACT | |
| B43 | LF | AACCCTGGCCCGAATGCTC | 47 |
| B43 | LB | GTCAACAATTCCTCCTCCTGCC | 48 |
| B44 | F3 | ACTCTTTGGAAGGCGGGTAT | 49 |
| B44 | B3 | TTGTTTGAGTTGGCTCCGAA | 50 |
| B44 | F1P | ACCAACCTCCCATGCTGTAGCT- | 51 |
| | | GAGAGAAACCACACGTAGCG | |
| B44 | B1P | CCTCGCAAAGGCATGGGGAC- | 52 |
| | | GGGTCCAACTGATGATCGG | |
| B44 | LF | CCCAAGAATATGGTGACCCGCAAA | 53 |
| B44 | LB | CCAACCCTCTGGGATTCTTTC | 54 |
| B45 | F3 | TCCCAACCCTCTGGGATTC | 55 |
| B45 | B3 | CACTGGGGTCAACATGCC | 56 |
| B45 | F1P | CCTTGATGGGGTTGAAGTCCCA- | 57 |
| | | AGTTGGACCCTGTATTCGGA | |
| B45 | B1P | GCCAGCAGCCAACCAGGTAG- | 58 |
| | | CTCCACCCCAAAACACCG | |
| BB | F3 | GGAGCCAACTCAAACAATCCAG | 59 |
| BB | B3 | GAGAGATGGGAGTAGGCTGTC | 60 |
| BB | F1P | GAACCCTGGCCCGAATGCTC TTTT | 61 |
| | | GCCAGAGGCAAATCAGGTAG | |
| BB | B1P | TGGAGCCCTCAGGCTCAGGG TTTTTT | 62 |
| | | ATTGGTGGAGGCAGGAGGAGG | |

### Example 2

### Detection of HBV in Mock Plasma Via LAMP and PCR Using Published Primer Set (Primer Set 2)

OPTIQUANT™ HBV DNA quantification panels was purchased from (AcroMetrix, Benicia, Calif.) and used as mock virus-positive plasma. Each OPTIQUANT™ HBV DNA panel member contains naturally occurring hepatitis B virus in human plasma at various concentrations, as shown in Table 2.

TABLE 2
Panel Reagents

| OptiQuant HBV Viral | | |
|---|---|---|
| DNA Quantification | HBV DNA | Quantity |
| Panel Member | Concentration (IU/mL) | (mL per vial) |
| OptiQuant negative | 0 | 0.5 |
| OptiQuant HBV 2E2 | 200 | 0.5 |
| OptiQuant HBV 2E3 | 2,000 | 0.5 |
| OptiQuant HBV E4 | 20,000 | 0.5 |
| OptiQuant HBV E5 | 200,000 | 0.5 |
| OptiQuant HBV E6 | 2,000,000 | 0.5 |
| OptiQuant HBV 2E7 | 20,000,000 | 0.5 |

100 μl of OptiQuant HBV 2E3, OptiQuant HBV 2E4 and OptiQuant HBV 2E5 mock plasma samples were heated at 125° C. for 15 min on a block heater (Lab-line Multi-block heater or Grant QBD2 block heater). After heated, 1 μl of treated sample was added into a 24 μl pre-mixed LAMP solution. The pre-mixed LAMP solution were prepared by mixing 1.2 μl stock solution of primers from primer set 2 (Table 3) (FIP and BIP 40 pmol, LF and LB 20 pmol, F3 and B3 5 pmol) with 12.5 μl 2× buffer (40 mM Tris.-HCl, 20 mM KCl, 16 mM MgSO₄, 20 mM [NH₄]₂SO₄, 0.2% TWEEN® 20, 1.6 M betaine and deoxynucleotides triphosphates 2.8 mM), and 1 μl Bsi DNA polymerase (8 unit/μl), and distilled water to a total of 24 μl. The sample mixture was then incubated at 60° C. for 60 or 90 minutes. 5 μl of 10×BlueJuice loading dye (INVITROGEN®, CA) was added to the sample mixture to stop the reaction. Five microliter of each sample was loaded on to agarose gel (2.5%) and allowed to run at 100V for 40 to 50 minutes (Embi Tec RunOne electrophoresis cell and power supply).

Mock plasma sample of 2×10⁵ IU/ml gave positive results after 60 minutes of incubation. Mock plasma sample of 2×10⁴ IU/ml also gave positive results after 90 minutes of incubation, but not after 60 minutes of incubation. This suggests that longer incubation time can increase the sensitivity of the assay. This experiment illustrated that pre-treated plasma without the step for nucleic acid extraction can be used directly as samples (template) in a LAMP assay. This improvement greatly simplified sample preparation. It posts significant advantage over the previous HBV LAMP assay (29), which required multiple steps to purify the nucleic acid template from, the blood sample before reaction.

TABLE 3
Published Primer Set for HBV Detection (Primer Set 2)

| Primer | Sequence 5′ to 3′ |
|---|---|
| F3-HBV-2 | CAAAATTCGCAGTCCCCAAC |
| B3-HBV-2 | GGTGGTTGATGTTCCTGGA |
| F1P-HBV-2 | GATAAAACGCCGCAGACACATCCTTCCAACCTCTTGTCCTCCAA |
| B1P-HBV-2 | CCTGCTGCTATGCCTCATCTTCTTTGACAAACGGGCAACATACCTT |
| LF-HBV-2 | CAGCGATAGCCAGGACAAA |
| LB-HBV-2 | GTTGGTTCTTCTGGACTACC |

### Example 3

### Detection of HBV in Mock Plasma Via LAMP Using Newly Designed Primer Set

100 μl of OptiQuant HBV 2E5 mock plasma samples were heated at 125° C. for 15 min. After the heat treatment, 1 μl of treated sample was added into a 24 μl LAMP pre-mixed LAMP solution. The LAMP pre-mixed LAMP solution were prepared by mixing 1.2 μl of stock solution for primers from each of the 12 primer sets (listed in table 1) with 12.5 μl 2× buffer (40 mM Tris-HCl, 20 mM KCl, 16 mM MgSO₄, 20 mM [NH₄]₂SO₄, 0.2% TWEEN® 20, 1.6 M betaine and deoxynucleotides triphosphates 2.8 mM), and 1 μl Bst DNA polymerase (8 unit/μl), and distilled water to a total of 24 μl. The sample mixture was then incubated at 60° C. for 60 minutes. 5 μl of 10×BlueJuice loading dye (INVITROGEN®, CA) was added to stop the reaction. 2 or 2.5% agarose gel was run at 100V for 40 to 50 minutes (Embi Tec RunOne electrophoresis cell and power supply). 9 (B11, B12, B13, BA, B41, B42, B43, B44, B45) primer sets can detect the presence of HBV in mock plasma sample of 2×10⁵ IU/ml.

### Example 4

### Detection of HBV in Mock Plasma Via LAMP Using Newly Designed Primer Set

100 μl of OptiQuant HBV 2E3 mock plasma samples were heated at 125° C. for 15 min. After the heat treatment, 1 μl of treated sample was added into a 24 μl LAMP pre-mixed LAMP solution. The LAMP pre-mixed LAMP solution were prepared by mixing 1.2 μl of stock solution of primers from each of eight selected primer sets (B11, B12, B13, BA, B42, B43, B44, B45) with 12.5 μl 2× buffer (40 mM Tris-HCl, 20 mM KCl, 16 mM MgSO₄, 20 mM [NH₄]₂SO₄, 0.2% TWEEN® 20, 1.6 M betaine and deoxynucleotides triphosphates 2.8 mM), and 1 μl Bst DNA polymerase (8 unit/μl), and distilled water to a total of 24 μl. The sample mixture was then incubated at 60° C. for 60 minutes. 5 μl of 10×BlueJuice loading dye (INVITROGEN®, CA) was added to stop the reaction. 2 or 2.5% agarose gel was run at 100V for 40 to 50 minutes (Embi Tec RunOne electrophoresis cell and power supply). 7 out of the 8 primer sets (B12, B13, BA, B42, B43, B44, B45) that gave positive results in example 3 can detect the presence of HBV from samples of a lower virus concentration of 2×10³ IU/ml. Primer 2 serves as a positive control for this experiment.

### Example 5

### Improve/Optimize the Limit of Detection (LOD)

100 μl of OptiQuant HBV 2E3 mock plasma samples were heated at 125° C. for 5, 10 or 15 minutes on a block heater (Lab-line Multi-block heater or Grant QBD2 block heater). After heat treatment, 1 or 4 μl of treated samples was added into a 24 μl or 21 μl of LAMP pre-mixed LAMP solution. The LAMP pre-mixed LAMP solution were prepared by mixing 1.2 μl of primers of primer set B43 (Table 1) (FIP and BIP 40 pmol, LF and LB 20 pmol, F3 and B3 5 pmol) with 12.5 μl 2× buffer (40 mM Tris-HCl, 20 mM KCl, 16 mM MgSO₄, 20 mM [NH₄]₂SO₄, 0.2% TWEEN® 20, 1.6 M betaine and deoxynucleotides triphosphates 2.8 mM), and 1 μl Bst DNA polymerase (8 unit/μl), and distilled water to a total of 24 μl. The sample mixture was then incubated at 60° C. for 60 minutes. 5 μl of 10×BlueJuice loading dye (INVITROGEN®, CA) was added to stop the reaction. 2 or 2.5% agarose eel was run at 100V for 40 to 50 min (Embi Tec RunOne electrophoresis cell and power supply). Increasing the amount of heated mock plasma (1 μl to 4 μl) or increasing the heating time (from 5 minutes to 15 minutes) is shown can both improve the sensitivity of the LAMP assay under otherwise same reaction conditions. Presence of HBV is not detected using 1 μl of mock plasma samples of 2×10³ IU/ml (lane 3), but 4 μl of the same mock plasma sample gave positive result (lane 4). Similarly, 1 μl of mock plasma sample of 2×10³ IU/ml heated for 5 minutes or 10 minutes pre-reaction did not yield any amplified products (lane 2 and lane 3), while same sample heated for 15 minutes clearly showed ample product.

### Example 6

### Fluorescence Labeled LAMP Detection of HBV

100 μl of OptiQuant HBV 2E3 mock plasma samples were heated at 125° C. for 15 min. After the heat treatment, 1 μl of treated sample was added into a 24 μl LAMP pre-mixed LAMP solution. The LAMP pre-mixed LAMP solution were prepared by mixing 1.2 μl of stock solution for primers from primer set B43 with 12.5 μl 2× buffer (40 mM Tris-HCl, 20 mM KCl, 16 mM MgSO₄, 20 mM [NH₄]₃SO₄, 0.2% TWEEN® 20, 1.6 M betaine and deoxynucleotides triphosphates 2.8 mM), and 1 μl Bst DNA polymerase (8 unit/μl), and distilled water to a total of 24 μl. The LB primer of B43 primer set is labeled with FAM. The sample mixture was then incubated at 60° C. for 60 minutes. 5 μl of BHQ-labeled complimentary sequence LB was added to the reaction mixture to quench the unincorporated FAM-labeled LB primer in the reaction mixture. The quenched reaction mixture was measured directly by a fluorescence tube scanner (ESEQuant TS) for 1 min and run 2 or 2.5% agarose gel at 100V for 40 to 50 minutes (Embi Tec RunOne electrophoresis cell and power supply) for comparison. FAM/BHQ labeled primer set B43 can detect the presence of HBV in mock plasma sample of 2×10³ IU/ml. The detection of fluorescence signal may be done either by fluorescence tube scanner or using agarose gel.

### Example 7

### Double Labeled LAMP Detection of HBV

100 μl of OptiQuant HBV 2E3 mock plasma samples were heated at 125° C. for 15 min. After the heat treatment, 1 μl or treated sample was added into a 24 μl LAMP pre-mixed LAMP solution. The LAMP pre-mixed LAMP solution were prepared by mixing 1.2 μl of stock solution for primers from primer set B43 with 12.5 μl 2× buffer (40 mM Tris-HCl, 20 mM KCl, 16 mM MgSO₄, 20 mM [NH₄]₂SO₄, 0.2% TWEEN® 20, 1.6 M betaine and deoxynucleotides triphosphates 2.8 mM), and 1 μl Bst DNA polymerase (8 unit/μl), and distilled water to a total of 24 μl. The LB primer of B43 primer set is labeled with FAM while the FIP primer is biotin labeled. The sample mixture was then incubated at 60° C. for 60 minutes. 5 μl of the reaction mixture was added to the loading area of an ICT assay strip. 150 μl of chasing buffer containing gold labeled rabbit anti-FAM was loaded to develop the signal. The strip was printed with streptavidin on the test line and anti-rabbit antibody on the control line. The results are read by eye in 15 minutes, which is shown in FIG. 3. FIG. 3 demonstrated that after LAMP reaction the signal can be visualized by naked eye without the use of additional instruments and procedure, such as running agarose gel, or using UV lamp box, and fluorescence tube scanner.

### REFERENCES

1. Spinella P C, Perkins J G, Grathwohl K W, Repine T, Beekley A C, Sebesta J, Jenkins D, Azarow K, Holcomb J B; 31st Combat Support Hospital Research Working Group. Risks associated with fresh whole blood and red blood cell transfusions in a combat support hospital. Crit Care Med. 2007 November: 35(11):2576-81. http://www.ncbi.nlm.nih.gov/pubmed/17828033?ordinalpos=3&itool=EntrezSystem2.PEntrez. Pubmed.Pubmed_ResultsPanel.Pubmed_DefaultReportPanel.Pubmed_RVDocSum
2. Spinella P C. Warm fresh whole blood transfusion for severe hemorrhage: U.S. military and potential civilian applications. Crit Care Med. 2008 July; 36(7 Suppl):S340-5. http://www.ncbi.nlm.nih.gov/pubmed/18594261?ordinalpos=1&itool=EntrezSystem2.PEntrez. Pubmed.Pubmed_ResultsPanel.Pubmed_DefaultReportPanel.Pubmed_RVDocSum
3. Comanor L, Holland P. Hepatitis B virus blood screening: unfinished agendas. Vox Sang. 2006 July; 91(1):1-12. http://www.ncbi.nlm.nih.gov/pubmed/16756595?ordinalpos=5&itool=EntrezSystem2.PEntrez. Pubmed.Pubmed_ResultsPanel.Pubmed_DefaultReportPanel.Pubmed_RVDocSum
4. Scott J D, Gretch D R. Molecular diagnostics of hepatitis C virus infection: a systematic review. JAMA. 2007 Feb. 21; 297(7):724-32. Available at: http://jama.ama-assn.org/cgi/content/full/297/7/724
5. Alter M J. Epidemiology of viral hepatitis and HIV co-infection. J. Hepatol. 2006;44(1 Suppl):S6-9. http://www.ncbi.nlm.nih.gov/pubmed/16352363?ordinalpos=7&itool=EntrezSystem2.PEntrez. Pubmed.Pubmed_ResultsPanel.Pubmed_DefaultReportPanel.Pubmed_RVDocSum
6. Kuhns M C, Busch M P. New strategies for blood donor screening for hepatitis B virus: nucleic acid testing versus immunoassay methods. Mol Diagn Ther. 2006; 10(2):77-91. http://www.ncbi.nlm.nih.gov/pubmed/16669606?ordinalpos=1&itool=EntrezSystem2.PEntrez. Pubmed.Pubmed_ResultsPanel.Pubmed_DefaultReportPanel.Pubmed_RVDocSum
7. Strainer S L. Current risks of transfusion-transmitted agents: a review. Arch Pathol Lab Med. 2007 May; 131(5):702-7. Available at: http://arpa.allenpress.com/arpaonline/?request=getdocument&doi=10.1043%2F1543-2165(2007)131%5B702:CROTAA%5D2.0.CO%3B2
8. World Health Organization. Hepatitis B [online]. Available at: http://www.who.int/csr/disease/hepatitis/whocdscsrlyo20022/en/index1.html
9. Hoofnagle J H. Hepatitis C: the clinical spectrum of disease. Hepatology. 1997 September; 26(3 Suppl 1):15S-20S. http://www.ncbi.nlm.nih.gov/pubmed/9305658?ordinalpos=2&itool=EntrezSystem2.PEntrez. Pubmed.Pubmed_ResultsPanel.Pubmed_DefaultReportPanel.Pubmed_RVDocSum
10. Hallam N F, Fletcher M L, Read S J, Majid A M, Kurtz J B, Rizza C R. Low risk of sexual transmission of hepatitis C virus. J. Med Virol. 1993 July; 40(3):251-3. http://www.ncbi.nlm.nih.gov/pubmed/7689092?ordinalpos=2&itool=EntrezSystem2.PEntrez. Pubmed.Pubmed_ResultsPanel.Pubmed_DefaultReportPanel.Pubmed_RVDocSum
11. Chung R T. Hepatitis C and B viruses: the new opportunists in HIV infection. Top HIV Med. 2006 June-July; 14(2):78-83. Available at: http://www.iasusa.org/pub/topics/2006/issue2/78.pdf
12. Saravanan S, Velu V, Kumarasamy N, Nandakumar S, Murugavel K G, Balakrishnan P, Suniti S, Thyagarajan S P. Coinfection of hepatitis B and hepatitis C virus in HIV-infected patients in south India. World J Gastroenterol. 2007 Oct. 7; 13(37):5015-20. http://www.ncbi.nlm.nih.gov/pubmed/17854146?ordinalpos=1&itool=EntrezSystem2.PEntrez. Pubmed.Pubmed_ResultsPanel.Pubmed_DefaultReportPanel.Pubmed_RVDocSum
13. Notomi T, Okayama H, Masubuchi H, Yonekawa T, Watanabe K, Amino N, Hase T. Loop-mediated isothermal amplification of DNA. Nucleic Acids Res. 2000 Jun. 15; 28(12):E63. Available at: http://www.pubmedcentral.nih.gov/articlerender.fcgi?tool=pubmed&pubmedid=108713 86
14. Grab D J, Lonsdale-Eccles J, Inoue N. Lamp for tadpoles. Nat Methods. 2005 September; 2(9):635; author reply 635-6.
15. Iwamoto T, Sonobe T, Hayashi K. Loop-mediated isothermal amplification for direct detection of Mycobacterium tuberculosis complex, M. avium, and M. intracellular in sputum samples. J Clin Microbiol. 2003 June; 41(6):2616-22. Available at: http://www.pubmedcentral.nih.gov/articlerenderfcgi?tool=pubmed&pubmedid=127918 88
16. Cai T, Lou G, Yang J, Xu D, Meng Z. Development and evaluation of real-time loop-mediated isothermal amplification for hepatitis B virus DNA quantification: A new tool for HBV management. J Clin Virol. 2008 April; 41(4):270-6. http://www.ncbi.nlm.nih.gov/pubmed/18296109?ordinalpos=1&itool=EntrezSystem2.PEntrez. Pubmed.Pubmed_ResultsPanel.Pubmed_DefaultReportPanel.Pubmed_RVDocSum
17. Iwata S, Shibata Y, Kawada J, Hara S, Nishiyama Y, Morishima T, Ihira M, Yoshikawa T, Asano Y, Kimura H. Rapid detection of Epstein-Barr virus DNA by loop-mediated isothermal amplification method. J Clin Virol. 2006 October; 37(2):128-33. http://www.ncbi.nlm.nih.gov/pubmed/16973412?ordinalpos=4&itool=EntrezSystem2.PEntrez. Pubmed.Pubmed_ResultsPanel.Pubmed_DefaultReportPanel.Pubmed_RVDocSum
18. Ihira M, Akimoto S, Miyake F, Fujita A, Sugata K, Suga S, Ohashi M, Nishimura N, Ozaki T, Asano Y, Yoshikawa T. Direct detection of human herpesvirus 6 DNA in serum by the loop-mediated isothermal amplification method. J Clin Virol. 2007 May; 39(1):22-6. http://www.ncbi.nlm.nih.gov/pubmed/17376739?ordinalpos=3&itool=EntrezSystem2.PEntrez. Pubmed.Pubmed_ResultsPanel.Pubmed_DefaultReportPanel.Pubmed_RVDocSumM
19. iyagawa J, Maeda H, Murauchi T, Kokeguchi S, Yamabe K, Tanimoto I, Nishimura F, Fukui K, Takashiba S. Rapid and simple detection of eight major periodontal pathogens by the loop-mediated isothermal amplification method. FEMS Immunol Med Microbiol. 2008 August; 53(3):314-21.
20. Hong T C, Mai Q L, Cuong D V, Parida M, Minekawa H, Notomi T, Hasebe F, Morita K. Development and evaluation of a novel loop-mediated isothermal amplification method for rapid detection of severe acute respiratory syndrome coronavirus. J Clin Microbiol. 2004 May; 42(5):1956-61. Available at: http://www.pubmedcentral.nih.gov/articlerenderfcgi?tool=pubmed&pubmedid=151311 54
21. Kurosaki Y, Takada A, Ebihara H, Grolla A, Kamo N, Feldmann H, Kawaoka Y, Yasuda J. Rapid and simple detection of Ebola virus by reverse transcription-loop-mediated isothermal amplification. J Virol Methods. 2007 April; 141(1):78-83. http://www.ncbi.nlm.nih.gov/pubmed/17194485?ordinalpos=2&itool=EntrezSystem2.PEntrez. Pubmed.Pubmed_ResultsPanel.Pubmed_DefaultReportPanel.Pubmed_RVDocSum
22. Soliman H, El-Matbouli M. Reverse transcription loop-mediated isothermal amplification (RT-LAMP) for rapid detection of viral hemorrhagic septicemia virus (VHS). Vet Microbiol. 2006 May 31; 114(3-4):205-13. http://www.ncbi.nlm.nih.gov/pubmed/16384659?ordinalpos=4&itool=EntrezSystem2.PEntrez. Pubmed.Pubmed_ResultsPanel.Pubmed_DefaultReportPanel.Pubmed_RVDocSum
23. Yoshida N, Fujino M, Ota Y, Notomi T, Nakayama T. Simple differentiation method of mumps Hoshino vaccine strain from wild strains by reverse transcription loop-mediated isothermal amplification (RT-LAMP). Vaccine. 2007 Jan. 26; 25(7):1281-6. http://www.ncbi.nlm.nih.gov/pubmed/17097200?ordinalpos=2&itool=EntrezSystem2.PEntrez. Pubmed.Pubmed_ResultsPanel.Pubmed_DefaultReportPanel.Pubmed_RVDocSum
24. Curtis K A, Rudolph D L, Owen S M. Rapid detection of HIV-1 by reverse-transcription, loop-mediated isothermal amplification (RT-LAMP). J Virol Methods. 2008 August; 151(2):264-70. http://www.ncbi.nlm.nih.gov/pubmed/18524393?ordinalpos=2&itool=EntrezSystem2.PEntrez. Pubmed.Pubmed_ResultsPanel.Pubmed_DefaultReportPanel.Pubmed_RVDocSum
25. McAvin J C, Powers M D, Blow J A, Putnam J L, Huff W B, Swaby J A. Deployable, field-sustainable, reverse transcription-polymerase chain reaction assays for rapid screening and serotype identification of dengue virus in mosquitoes. Mil Med. 2007 March; 172(3):329-34. http://www.ncbi.nlm.nih.gov/pubmed/17436782?ordinalpos=1&itool=EntrezSystem2.PEntrez. Pubmed.Pubmed_ResultsPanel.Pubmed_DefaultReportPanel.Pubmed_RVDocSum
26. Wu S J, Pal S, Ekanayake S, Greenwald D, Lara S, Raviprakash K, Kochel T, Porter K, Hayes C, Nelson W, Callahan J. A dry-format field-deployable quantitative reverse transcriptase-polymerase chain reaction assay for diagnosis of dengue infections. Am J Trop Med Hyg. 2008 October; 79(4):505-10. Available at: http://www.ajtmh.org/cgi/content/full/79/4/505
27. Mori Y, Nagamine K, Tomita N, Notomi T. Detection of loop-mediated isothermal amplification reaction by turbidity derived from magnesium pyrophosphate formation. Biochem Biophys Res Commun. 2001 Nov. 23; 289(1):150-4. http://www.ncbi.nlm.nih.gov/pubmed/11708792?ordinalpos=3&itool=EntrezSystem2.PEntrez. Pubmed.Pubmed_ResultsPanel.Pubmed_DefaultReportPanel.Pubmed_RVDocSum
28. Tomita N, Mori Y, Kanda H, Notomi T. Loop-mediated isothermal amplification (LAMP) of gene sequences and simple visual detection of products. Nat Protoc. 2008; 3(5):877-82. http://www.ncbi.nlm.nih.gov/pubmed/18451795?ordinalpos=1&itool=EntrezSystem2.PEntrez. Pubmed.Pubmed_ResultsPanel.Pubmed_DefaultReportPanel.Pubmed_RVDocSum
29. Nagamine K, Hase T, Motomi T. Accelerated reaction by loop-mediated isothermal amplification using loop primers. Molecular and Cellular Probes 2002 16:223-229.
30. Horisaka T, Fujita K, Iwata T., Nakadai A, Okatani A T, Horikita T., Taniguchi T, Honda E., Yokomizo Y, and Hayashidani H. Sensitive and Specific Detection of Yersinia pseudotuberculosis by Loop-Mediated Isothermal Amplification. J Clin Microbiol. 2004 November; 42(11): 5349-5352.
31. Poon L L M, Wong B W Y, Ma E H T, Chan K H, Chow L M C, Abeyewickreme W, Tangpukdee N, Yuen K Y, Guan Y, Looareesuwan S and Peiris J S M. Sensitive and Inexpensive Molecular Test for Falciparum Malaria: Detecting Plasmodium falciparum DNA Directly from Heat-Treated Blood by Loop-Mediated Isothermal Amplification. 2006. Clinical Chemistry 52: 303-306.
32. Kay, A.; Zoulim, F. Hepatitis B virus genetic variability and evolution. Virus research. 2007. 127 (2): 164-176.

What is claimed is:

1) A method for detecting a Hepatitis B Virus in blood comprising:
(a) collecting a blood sample from a subject;
(b) heating said blood sample for a period of time;
(c) adding said heated sample to a pre-mixed LAMP solution creating a reaction mixture, wherein said pre-mixed LAMP solution comprising: one or more sets of LAMP primers and list DNA polymerase.
(d) incubating said reaction mixture for a period of time; and
(e) detecting the presence of HBV.

2) The method of claim 1, wherein said blood sample is whole blood, serum or plasma.

3) The method of claim 1, wherein said blood sample is heated at approximately 90° C. -135° C. in step (b).

4) The method of claim 3, wherein said blood sample was heated at approximately 115° C. -125° C. in step (b).

5) The method of claim 1, wherein said blood sample was heated for approximately 5-20 minutes in step (b).

6) The method of claim 1, wherein said reaction mixture is incubated at approximately 50° C. -80° C. in step (d).

7) The method of claim 1, wherein said reaction mixture is incubated at a reaction pH of approximately 6.0-9.5 in step (d).

8) The method of claim 1, wherein said reaction mixture is incubated for at least 15 minutes in step (d).

9) The method of claim 1, wherein said LAMP primer set is selected from the group consisting of primer sets B11, B12, B13, B14, B15, BA, B41, B42, B43, B44, B45, BB, and primer set 2.

10) The method of claim 1, wherein approximately 1-10 μl of said heated sample is added to the pre-mixed LAMP solution.

11) The method of claim 1, further comprising adding a dye or a fluorescent metal indicator to said reaction mixture.

12) The method of claim 11, wherein said dye comprising SYBR Green, or Manganese ion and calcein.

13) The method of claim 1, wherein a fluorescent molecule linked to 5′ terminus of a primer of said primer set.

14) The method of claim 13, wherein step (e) further comprising:
i. adding a quencher probe to said reaction mixture after incubation step, said quencher probe is a complementary nucleotide sequence of said selected primer that is linked to a quencher on its 3′ end; and
ii. detecting the presence of HBV virus by measuring fluorescence of the reaction mixture.

15) The method of claim 13, wherein said fluorescent molecule is FAM.

16) The method of claim 15, wherein said quencher probe is BHQ.

17) The method of claim 1, wherein a first primer from said primer set is labeled with a first tag and a second primer from said primer set is labeled with a second tag.

18) The method of claim 17, step (e) further comprising:
i. adding the reaction mixture onto a LAMP-ICT strip;
ii. adding flowing buffer to said LAMP-ICT strip; and
iii. detecting the presence of HBV in said blood sample.

19) The method of claim 18, wherein said LAMP-ICT strip comprising a test region capable of binding to the first tag.

20) The method of claim 19, wherein said LAMP-ICT strip further comprising a region upstream from the test region which is coated with a label particle capable of binding to the second tag.

21) The method of claim 19, wherein said a control region downstream from said test region.

22) A reagent for detection of Hepatitis B using loop-mediated isothermal amplification comprising:
a. one or more LAMP primer set for Hepatitis B; and
b. a Bst DNA polymerase.

23) The reagent of claim 22, wherein said Hepatitis B primer set is selected from the group consisting of primer sets B11, B12, B13, B14, B15, BA, B41, B42, B43, B44, B45, and BB.

24) The reagent of claim 22, further comprising a buffer.

25) The reagent of claim 24, wherein said buffer has a pH of 6.0-9.5.

26) The reagent of claim 22, further comprising a fluorescent metal indicator.

27) The reagent of claim 26, wherein a fluorescence molecule is linked to 3′ terminus of a primer selected said primer set.

28) The reagent of claim 27, wherein a first primer from said primer set is labeled with FAM and a second primer is labeled with biotin.
